(19) United States
(12) Patent Application Publication
(10) Pub. No.: US 2026/0102565 A1
(43) Pub. Date: Apr. 16, 2026

(54) Symmetrical Sequential Stopper for a Multi-Chamber Medical Injection Device

(71) Applicant: Becton, Dickinson and Company, Franklin Lakes, NJ (US)

(72) Inventors: Marc Flippe, Claix (FR); Simon Rempfer, St Hilaire de la Cote (FR); Julien Singer, Domène (FR); Ferdinand Lavigne, Seyssinet-Pariset (FR); Jimmy Voniez, Grenoble (FR)

(21) Appl. No.: 19/265,617

(22) Filed: Jul. 10, 2025

Related U.S. Application Data
(60) Provisional application No. 63/670,296, filed on Jul. 12, 2024.

(51) Int. Cl. A61M 5/19; A61M 5/31

(52) U.S. Cl. CPC A61M 5/19; A61M 5/3129

(57) ABSTRACT

Provided herein is a sequential stopper positionable within a barrel of an injection device. The sequential stopper includes a membrane comprising a proximal face and a distal face and that separates a distal chamber and a proximal chamber of the barrel. The membrane includes a slit extending therethrough that selectively creates a fluid path through the membrane when a sufficient fluid pressure is exerted onto the proximal fac thereof, thereby transferring fluid from the proximal chamber to the distal chamber. A lateral wall is joined to the membrane that defines proximal and distal cavities and includes ribs that sealingly engage an inner surface of the barrel. The sequential stopper is formed as a symmetrical stopper where the proximal face and the distal face have a corresponding configuration and where the membrane is positioned intermediate between a distal end and a proximal end of the sequential stopper.

### CROSS-REFERENCE TO RELATED APPLICATION

The present application claims priority to U.S. Provisional Patent No. 63/670,296 entitled “Symmetrical Sequential Stopper for a Multi-Chamber Medical Injection Device” filed Jul. 12, 2024, the entire disclosure of which is hereby incorporated by reference.

### BACKGROUND OF THE INVENTION

### Field of the Invention

The present disclosure relates generally to a sequential stopper for a medical injection device containing doses of multiple fluids and, in particular, to a sequential stopper for a medical injection device that provides sequential injection of a first or initial fluid, such as a first type of a medical fluid, followed by a secondary fluid, with such fluids being of the same type or different types.

### Description of Related Art

Prefilled injection devices are common containers used to administer liquids (e.g., medications or drugs) to a patient and include syringes, cartridges and auto-injectors or the like. They usually comprise a plunger stopper in gliding engagement within a container, the container being filled with a pharmaceutical composition in order to provide the practitioners with a ready-to-use injection device for patients.

A container has a substantially cylindrical shape and comprises a proximal end able to be stoppered by a plunger stopper, a distal end wherein the pharmaceutical composition is expelled from the container, and a lateral wall extending between the proximal end and the distal end of the container. In practice, the plunger stopper is aimed at moving, upon the pressure exerted by a plunger rod, from a proximal end of the container towards the distal end of the container, thereby expelling the drug contained into the container.

When compared to empty injection devices that are filled with a vial-stored pharmaceutical composition just prior to the injection to the patient's body, the use of prefilled injection devices leads to several advantages. In particular, by limiting the preparation prior to the injection, the prefilled injection devices provide a reduction of medical dosing errors, a minimized risk of microbial contamination and an enhanced convenience of use for the practitioners. Furthermore, such prefilled containers may encourage and simplify self-administration by the patients which allows reducing the cost of therapy and increasing the patient adherence. Finally, prefilled injection devices reduce loss of valuable pharmaceutical composition that usually occurs when a pharmaceutical composition is transferred from a vial to a non-prefilled injection device. This results in a greater number of possible injections for a given manufacturing batch of pharmaceutical composition, thus reducing buying and supply chain costs.

Prefilled injection devices can be used to carry out the injection of a plurality of compositions or medicaments to a patient. In such case, the container may include two chambers, including a first chamber adapted to contain a first composition and a second chamber adapted to contain a second composition. The two chambers are separated by a second stopper that may be termed as a sequential stopper that prevents, when the prefilled injection devices is stored or transported, the compositions from passing from one chamber to the other and mixing.

In some prefilled injection devices that include multiple compositions for injection to a patient, the barrel of the injection device may be modified (from a typical cylindrical barrel) to include one or more bypass channels therein. The bypass channels may provide for a reconstitution of a lyophilized composition (with a composition first flowing through the bypass channel) and a subsequent injection of the reconstituted mixture and/or provide for a sequential injection of the compositions in the barrel (via initial injection of a first/distal composition and a subsequent injection of a second/proximal composition that flows through the bypass (when the stopper is moved distally past the bypass channel). However, it is recognized that the manufacturing of a barrel to include such bypass channels can greatly increase the manufacturing or packaging costs thereof.

In other prefilled injection devices that include multiple compositions for injection to a patient, the sequential stopper includes an opening or slit formed therein or therethrough that functions as a valve (i.e., a “sequential stopper”) to prevent the mixing of the two solutions during storage or transportation, or before the first solution has been injected, while allowing subsequent injection of the second solution. Existing sequential stoppers function to effectively prevent the mixing of the two solutions and provide for a sequential injection of the fluids; however, existing sequential stoppers do have a number of drawbacks associated therewith. As one example, the configuration of the sequential stopper may be such that a substantial force may need to be applied onto the plunger to force the second solution through the opening/slit therein and expelling it out the injection device. As another example, the structure of the sequential stopper may be complex—such as being configured as a multi-component stopper where a valve is separate from a stopper body—which may be detrimental for the functioning of the valve assembly and/or its manufacturing. As still further examples, the design of existing sequential stoppers may lead to issues with opening the valve smoothly, providing a desired flux or flowrate through the stopper, and/or maintaining valve robustness or integrity until injection.

Accordingly, a need exists in the art for an injection device that includes a typical cylindrical barrel and a sequential stopper that addresses the aforementioned drawbacks.

### SUMMARY OF THE INVENTION

Provided herein is a sequential stopper configured to be positioned inside a barrel of a multi-chamber injection device for injecting at least one fluid through a distal end of the barrel. The sequential stopper includes a membrane comprising a proximal face and a distal face, the membrane being configured to separate a first, distal chamber of the barrel from a second, proximal chamber of the barrel, with the membrane including a slit extending therethrough between the proximal face and the distal face, with the slit configured to selectively create a fluid path through the membrane from the proximal face to the distal face depending on the pressure exerted by a composition onto the proximal face of the membrane for transferring fluid only from the proximal chamber to the distal chamber. The sequential stopper also includes a lateral wall joined to the membrane to define a distal cavity and a proximal cavity, the lateral wall comprising a sealing surface including a plurality of circumferential ribs configured to sealingly engage the inner surface of the barrel. The sequential stopper comprises a symmetrical stopper where the proximal face and the distal face have a corresponding configuration and wherein the membrane is positioned intermediate between a distal end and a proximal end of the sequential stopper.

In certain configurations, each of the proximal face and the distal face has a flat shape, a convex shape, a concave shape, or a multi-shape profile.

In certain configurations, the plurality of circumferential ribs comprises a first rib extending radially outward from an outer surface of the lateral wall and around an outer circumference thereof, the first rib comprising a distal rib, and a second rib extending radially outward from the outer surface of the lateral wall and around the outer circumference thereof, the second rib comprising a proximal rib, wherein the first rib is spaced apart lengthwise from the second rib by an inter-rib region of the lateral wall.

In certain configurations, the inter-rib region comprises a curved inter-rib region, where an outer surface of the lateral wall is curved radially inward.

In certain configurations, the curved inter-rib region has a radius of curvature of 1 mm to 10 mm.

In certain configurations, the radius of curvature is between 2 mm and 3 mm.

In certain configurations, an inner surface of the lateral wall joins with the membrane via connecting portions therebetween, with the connecting portions providing a hinge about which the membrane deflects or pivots when pressure is exerted onto the proximal face of the membrane by the composition that causes the slit to open.

In certain configurations, a lengthwise region between the proximal rib and the membrane and between the distal rib and the membrane comprises a force transfer area, where a radially inward-directed compressive force from the syringe barrel pressing against the first and second ribs is transferred to the membrane, to keep the slit closed under radial compression.

In certain configurations, an angle of force transfer from the first and second ribs to the membrane, via the lateral wall and connecting portions, is between 20 degrees and 60 degrees, and preferably between 30 degrees and 45 degrees.

In certain configurations, a thickness of the lateral wall and connecting portions in the force transfer area is between 0.5 mm and 2 mm.

In certain configurations, the plurality of ribs comprises a first rib extending radially outward from an outer surface of the lateral wall and around an outer circumference thereof, the first rib comprising a distal rib, a second rib extending radially outward from the outer surface of the lateral wall and around the outer circumference thereof, the second rib comprising a proximal rib, and a third rib extending radially outward from the outer surface of the lateral wall and around the outer circumference thereof, the third rib comprising a center rib, wherein the membrane is aligned with the third rib lengthwise along the sequential stopper.

In certain configurations, the first rib and the second rib have a first outer diameter and the third rib has a second outer diameter that is smaller than the first outer diameter.

In certain configurations, the membrane comprises one or more gutters formed therein, in the proximal face and/or the distal face of the membrane, with the one or more gutters reducing the thickness of the membrane at a location thereof, and wherein the membrane deflects or pivots about the one or more gutters when pressure is exerted onto the proximal face of the membrane by the composition that causes the slit to open.

In certain configurations, the sequential stopper is a mono-material stopper, with the membrane and lateral wall formed as a single molded component.

In certain configurations, the sequential stopper is a bi-material stopper, with the membrane formed of a first material and the lateral wall formed of a second material.

Also provided herein is a medical injection device for injecting at least one fluid. The medical injection device comprises a barrel extending from a proximal end to a distal end, with the barrel comprising a cylindrical wall, an end wall positioned at the distal end, and a tip extending distally from the end wall. The medical injection device further comprises a plunger stopper adapted to be translationally movable inside the barrel, and a sequential stopper arranged between the distal end of the barrel and the plunger stopper, and adapted to be translationally movable inside the barrel. The sequential stopper comprises a membrane having a proximal face and a distal face, with the membrane being configured to separate a first, distal chamber of the barrel from a second, proximal chamber of the barrel. The sequential stopper further comprises a lateral wall joined to the membrane, the lateral wall comprising a circumferential sealing surface configured to sealingly engage the inner surface of the barrel. A first chamber is defined within the barrel between the distal end of the barrel and the sequential stopper and a second chamber is defined within the barrel between the sequential stopper and the plunger stopper.

In certain configurations, the medical injection device is configured to sequentially inject multiple fluids, such as two fluids sequentially injected, with a first fluid contained within the first chamber and a second fluid contained within the second chamber, and wherein the sequential stopper is spaced apart distally from the end wall to separate the first chamber from the second chamber.

In certain configurations, the sequential stopper comprises a first sequential stopper, and the medical injection device further comprises a second sequential stopper, the second sequential stopper identical to the first sequential stopper, with the first sequential stopper and the second sequential stopper in part defining a first chamber, a second chamber, and a third chamber in the barrel.

In certain configurations, wherein the medical injection device is configured to sequentially inject three fluids, with a first fluid contained within the first chamber, a second fluid contained within the second chamber, and a third fluid contained within the third chamber.

In certain configurations, the medical injection device is one of a syringe or a cartridge.

### BRIEF DESCRIPTION OF THE DRAWINGS

FIG. 1 is a perspective view of a multi-chamber medical injection device, according to a non-limiting embodiment described herein;

FIG. 2 is an exploded view of the multi-chamber medical injection device of FIG. 1;

FIG. 3 is a perspective view of a sequential stopper for use in a medical injection device, according to a non-limiting embodiment described herein;

FIG. 4 is a cross-sectional view of the sequential stopper of FIG. 3;

FIG. 5 is a cross-sectional view of a sequential stopper for use in a medical injection device, according to another non-limiting embodiment described herein;

FIG. 6 is a cross-sectional view of a sequential stopper for use in a medical injection device, according to another non-limiting embodiment described herein;

FIG. 7 is a cross-sectional view of a sequential stopper for use in a medical injection device, according to another non-limiting embodiment described herein;

FIG. 8 is a cross-sectional view of a sequential stopper for use in a medical injection device, according to another non-limiting embodiment described herein;

FIG. 9 is a cross-sectional view of a sequential stopper for use in a medical injection device, according to another non-limiting embodiment described herein;

FIG. 10 illustrates the sequential stopper of FIGS. 3 and 4 with compressive forces being applied to the sequential stopper;

FIG. 11 illustrates the sequential stopper of FIGS. 3 and 4 with a distally directed fluid pressure being applied to the sequential stopper, along with a resulting deflection of the sequential stopper;

FIG. 12 is a cross-sectional view of a sequential stopper for use in a medical injection device, according to another non-limiting embodiment described herein;

FIG. 13 illustrates the sequential stopper of FIG. 12 with a distally directed fluid pressure being applied to the sequential stopper, along with a resulting deflection of the sequential stopper;

FIG. 14 is a cross-sectional view of a sequential stopper for use in a medical injection device, according to another non-limiting embodiment described herein;

FIG. 15 is a cross-sectional view of a sequential stopper for use in a medical injection device, according to another non-limiting embodiment described herein;

FIG. 16 illustrates the sequential stopper of FIG. 14 with compressive forces being applied to the sequential stopper;

FIG. 17 is a cross-sectional view of a bi-material sequential stopper for use in a medical injection device, manufactured via an overmolding process, according to another non-limiting embodiment described herein;

FIG. 18 is a cross-sectional view of a bi-material sequential stopper for use in a medical injection device, manufactured via an assembly process, according to another non-limiting embodiment described herein;

FIGS. 19A-19D illustrate various configurations of the injection device of FIG. 1 during injection of first and second compositions from the device;

FIGS. 20A-20C illustrate various configurations of an injection device during injection of a single composition from the device, according to another non-limiting embodiment described herein;

FIG. 21 is a cross-sectional view of a multi-chamber medical injection device, according to another non-limiting embodiment described herein;

FIG. 22 is a cross-sectional view of a multi-chamber medical injection device, according to another non-limiting embodiment described herein;

FIG. 23 is a cross-sectional view of a multi-chamber medical injection device, according to another non-limiting embodiment described herein; and

FIG. 24 is a cross-sectional view of a multi-chamber medical injection device, according to another non-limiting embodiment described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a syringe or other injection device in preparation for or during use. Similarly, in this application, the terms “in the distal direction” and “distally” mean in the direction toward the distal tip of the syringe, and the terms “in the proximal direction” and “proximally” mean in the direction opposite the direction of the distal tip of the syringe.

With reference to the figures, the present disclosure is directed to a sequential stopper 10 (alternately, a “valve stopper”) for use with a multi-chamber medical injection device 100, with it understood that the term “injection device” as used herein is meant to refer to a syringe used for a direct injection (“naked syringe”), or to a syringe or cartridge that may be used with an injection device (i.e., an auto-injector or pen, or another safety device), as non-limiting examples. The sequential stopper 10 is configured to divide a container, such as a syringe barrel or cartridge, into a distal chamber, which contains a first or initial fluid to be injected to a patient, and a proximal chamber, which contains a subsequent or secondary fluid to be delivered to the patient after the initial fluid. The sequential stopper 10 comprises a slit therein that functions as a built-in check valve that allows the secondary fluid contained in the proximal chamber of the injection device 100 to pass through the sequential stopper 10 only after the initial fluid is expelled from a distal chamber of the injection device 100. Accordingly, the multi-chamber injection device 100 of the present disclosure can be used for fluid delivery of multiple medical fluids through a needle cannula inserted into a patient with only one needle stick and no additional fluid delivery steps to be performed by the practitioner. In other examples, the multi-chamber injection device 100 of the present disclosure can allow for delivery of multiple medical fluids to a patient in sequence through a VAD without needing to attach multiple syringes or fluid containers to the VAD.

As described in further detail herein, the sequential stopper 10 is structured to prevent mixing of the two fluids before complete injection of the first fluid, while also allowing for injection of the second solution through the sequential stopper without requiring an excessive force to be applied to a proximal face of the sequential stopper to open the slit therein. As an example, the sequential stopper 10 is constructed such that an amount of force that must be applied to a plunger rod of the injection device to open the slit of the sequential stopper may be reduced, as compared to existing multi-chamber medical injection devices that include a sequential stopper.

The present disclosure is also directed to features of a prefilled multi-chamber injection device 100 including the sequential stopper 10. In particular, the multi-chamber injection device 100 can be configured to expel the initial fluid followed by the secondary fluid from a syringe barrel or cartridge through a fluid port or nozzle of the injection device 100. As previously described, the initial fluid can be a medical fluid, which, as used herein, can refer to a medication or another therapeutic agent used for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect. The secondary fluid can be another medical fluid, such as another type of therapeutic agent or drug. The secondary fluid can also be a flush solution, such as saline, a heparin lock flush solution, or another flush solution, as are known in the art.

The injection device 100 of the present disclosure allows a practitioner, such as a medical technician, nurse, physician assistant, physician, or other trained or untrained clinicians or a patient themselves (when used with an auto-injector), to administer the initial fluid followed by the secondary fluid without needing to change syringes or fluid containers between delivery of the initial fluid and the secondary fluid. Further, the injection device 100 of the present disclosure allows the practitioner to provide the sequential delivery of the initial fluid followed by the secondary fluid through a single continuous advancement of a plunger rod of the injection device 100. As used herein, “single continuous advancement of a plunger rod” means that the practitioner is able to push the plunger rod in a distal direction, through the barrel, as a single continuous stroke to expel the initial fluid followed by the secondary fluid from the syringe barrel. The practitioner does not need, for example, to perform multiple needle sticks or to disconnect a syringe or another device from the VAD between delivery of the initial fluid and the secondary fluid. Further, using the injection device 100 of the present disclosure, the practitioner does not need to perform any other action, such as twisting, rotating, or pulling on the plunger rod or pressing another component or mechanism of the injection device 100, in order to perform the sequential delivery of the initial fluid and the secondary fluid. Accordingly, the fluids can be expelled from the injection device 100 in sequence in response solely to the single continuous stroke of the plunger rod in the distal direction by the practitioner, which can be performed as a “single-handed” operation or movement (i.e., the practitioner can hold the injection device 100 and press the plunger rod through the barrel with one hand). Accordingly, the injection device 100 of the present disclosure simplifies processes for administering the initial fluid followed by the secondary fluid to a VAD and/or patient compared to conventional fluid delivery practices.

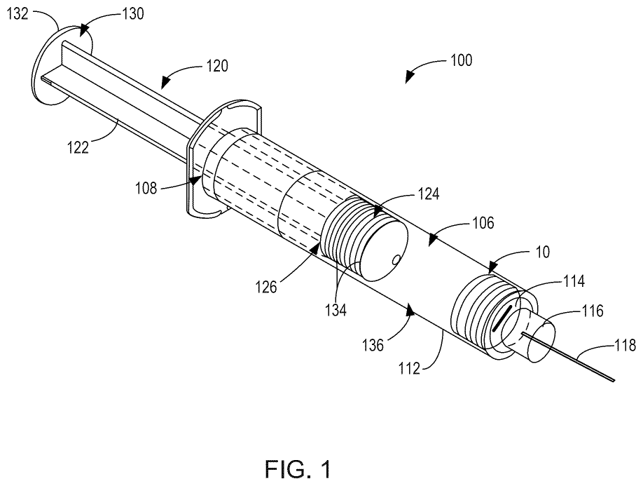

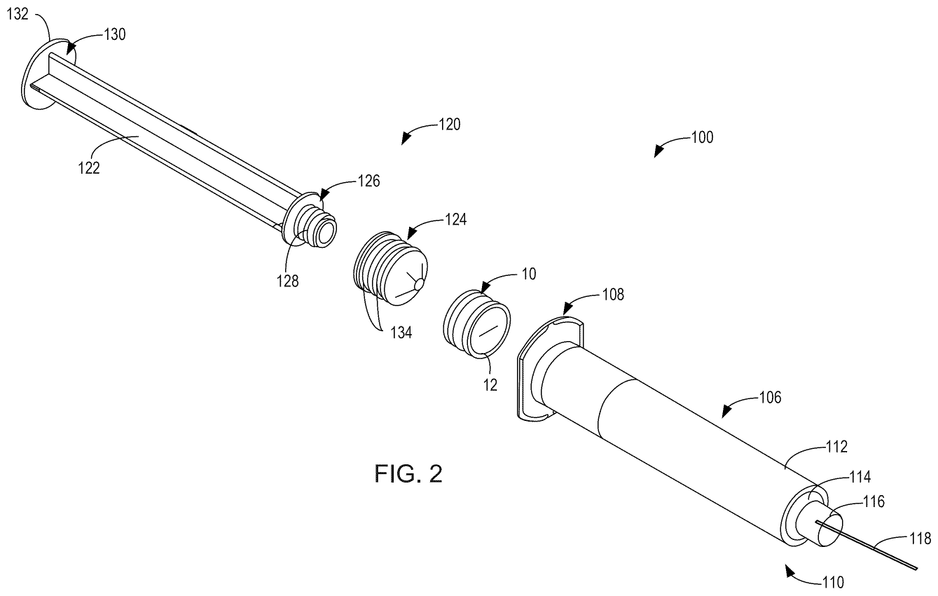

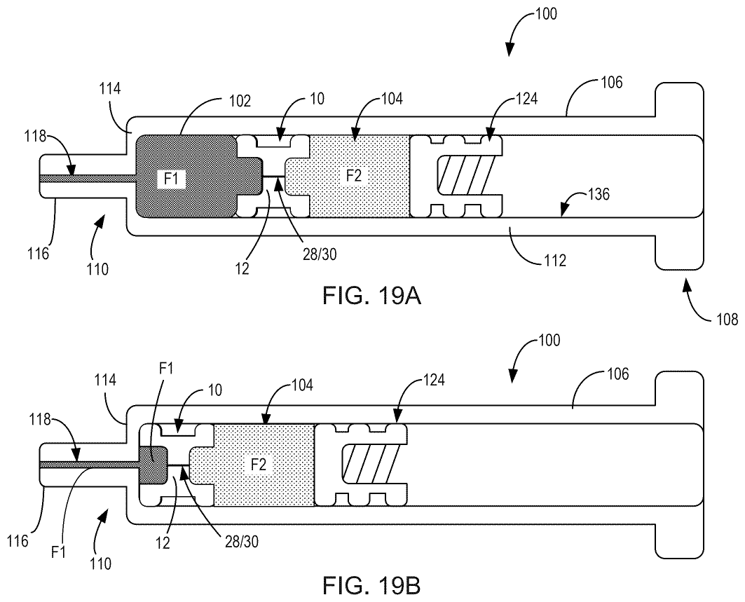

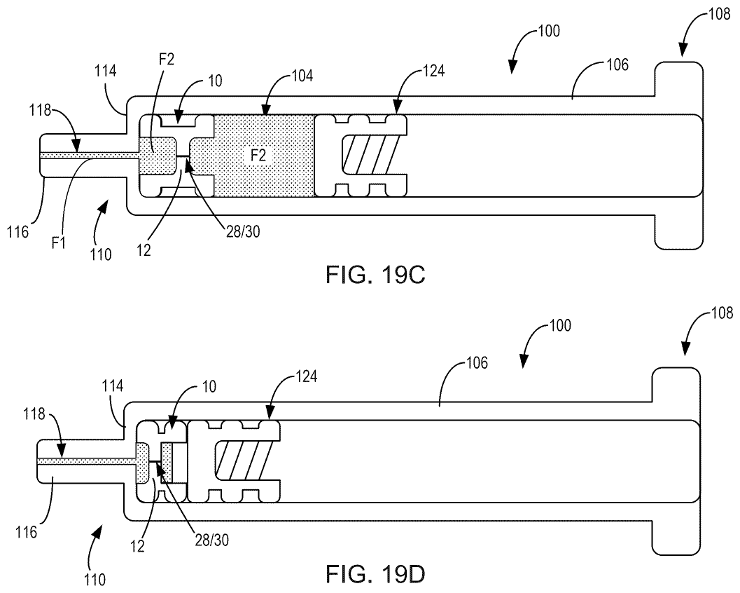

FIGS. 1 and 2 illustrate an example of a multi-chamber injection device 100 for sequential expulsion of at least an initial fluid F1 contained in a first or distal fluid chamber 102 (shown in FIGS. 19A and 19B) followed by a secondary fluid F2 contained in a second or proximal fluid chamber 104 (shown in FIGS. 19A-19D). In the illustrated embodiment, the injection device 100 is provided as a syringe, and is thus referred to hereafter as “syringe 100”; however, it is recognized that the injection device could also be provided as a cartridge useable in a pen, according to another non-limiting embodiment.

As previously described, the initial fluid F1 can be a medical fluid, such as a drug or another therapeutic agent intended for delivery to a patient through a needle cannula or through a VAD, such as a catheter or IV line. The secondary fluid F2 can be another type of therapeutic agent, or a flush solution such as saline solution, and/or an anticoagulant such as heparin. The first fluid F1 and second fluid F2 may have similar or dissimilar properties, including a concentration, viscosity, and/or pressure of the fluids, as non-limiting examples, and the fluid in each chamber could be with a different volume injected in term of dose delivered. The type and amount of solution contained in the proximal chamber 104 and/or the distal chamber 102 may vary depending, for example, on the specific type of needle cannula, catheter, or IV line being used for an injection and/or on the therapeutic effect to be achieved. In some examples, the syringe 100 contains or is configured to contain between about 0.1 mL and 20 mL of the initial fluid F1 and/or the secondary fluid F2.

In some examples, the syringe 100 comprises a barrel 106 having an open proximal end 108 and a distal end 110. The barrel 106 may be formed of a cylindrical sidewall 112 extending between the proximal end 108 and the distal end 110, along with an end wall 114 and a tip 116 at the distal end 110. The tip 116 may include a channel (not shown) formed therein within which a needle cannula 118 is secured, with the needle 118 providing for injection of the initial fluid F1 and the secondary fluid F2 from the barrel 106. In other embodiments, the tip 116 may be configured as a needleless connector configured to be connected directly or indirectly to a fluid port, valve, or another terminal access portion of a vascular access device (VAD).

The syringe 100 further comprises a plunger assembly 120 that includes a plunger rod 122 and a plunger stopper 124. The plunger rod 122 can be a conventional plunger rod used in currently available syringes. The plunger rod 122 can be, for example, an injection molded part formed from a rigid thermoplastic material, such as polyester, polycarbonate, polypropylene, polyethylene, polyethylene terephthalate, or another thermoplastic material, or may be formed of wood or metal, as are known in the art. In some embodiments, the plunger rod 122 can be connected to the plunger stopper 124 by mechanical connectors, threads, fasteners, or adhesives, while in other examples the plunger rod 122 can be integrally formed or co-molded with the plunger stopper 124. In an exemplary embodiment, the plunger rod 122 includes a distal end 126 engaged to the plunger stopper 124. For example, as most clearly seen in FIG. 2, the distal end 126 of the plunger rod 122 can include a threaded connector 128 that is inserted into a corresponding cavity (not shown) extending inwardly from a proximal surface of the plunger stopper 124. The plunger rod 122 also includes a proximal end 130 protruding proximally from the proximal end 108 of the syringe barrel 106. The proximal end 130 of the plunger rod 122 can include a thumb press plate 132 for manipulating the plunger rod 122 to move the plunger stopper through the syringe barrel 106.

The plunger stopper 124 may include many features of conventional syringe stoppers or plungers, as are known in the art. That is, the plunger stopper 124 can be a substantially cylindrical body formed from a flexible and/or deformable material, such as an elastomer, or a thermoplastic elastomer material. Examples of elastomers and thermoplastic elastomers include, but are not limited to, silicone or synthetic or natural rubber (e.g., isoprene), or combinations thereof. The plunger stopper 124 can include radially extending ribs or rings 134 that seal against an inner surface 136 of the syringe barrel 106 so that the plunger stopper 124 can move fluids through the syringe barrel 106 towards the distal end 110 of the barrel 106. In some examples, the plunger stopper 124 includes one or multiple annular ribs 134 (e.g., at least one, two or three ribs) in order to improve stability and to prevent the plunger stopper 124 from tilting, shifting, or otherwise deforming as the plunger stopper 124 moves through the syringe barrel 106, and to prevent microbial contamination of the internal volume of the barrel.

As indicated above, the syringe 100 further comprises a sequential stopper 10 slidably positioned within the barrel 106 of the syringe 100. The sequential stopper 10 separates the barrel 106 into the proximal chamber 104 and the distal chamber 102. Specifically, as shown most clearly in FIG. 19A, the proximal chamber 104 is between a distal end of the plunger stopper 124 and a proximal end of the sequential stopper 10 and the distal chamber 102 is between a distal end of the sequential stopper 10 and the distal end 110 of the barrel 106.

According to aspects of the disclosure, the sequential stopper 10 is structured to prevent mixing of the second fluid initially contained in the proximal chamber 104 with the first fluid initially contained in the distal chamber 102 before complete injection of the first fluid. The sequential stopper 10 may be moved distally through the barrel 106 responsive to a distal movement of the plunger stopper 124, with the sequential stopper 10 moving in coordination with the plunger stopper 124, even though the stoppers 10 and 124 are not mechanically connected or engaged together. The sequential stopper 10 further enables injection of the second fluid through the sequential stopper 10 without requiring an excessive force to be applied to the plunger rod 122, as explained in further detail below. The sequential stopper 10 may function as a one-way check valve for selectively controlling fluid flow between the proximal chamber 104 and the distal chamber 102. As used herein, a one-way check valve refers to a valve that allows a flow of fluid through the valve in only one direction. For example, when positioned within syringe barrel 106, the sequential stopper 10 operates to permit fluid flow from the proximal chamber 104 to the distal chamber 102, while fluid flow from the distal chamber 102 to the proximal chamber 104 is prevented.

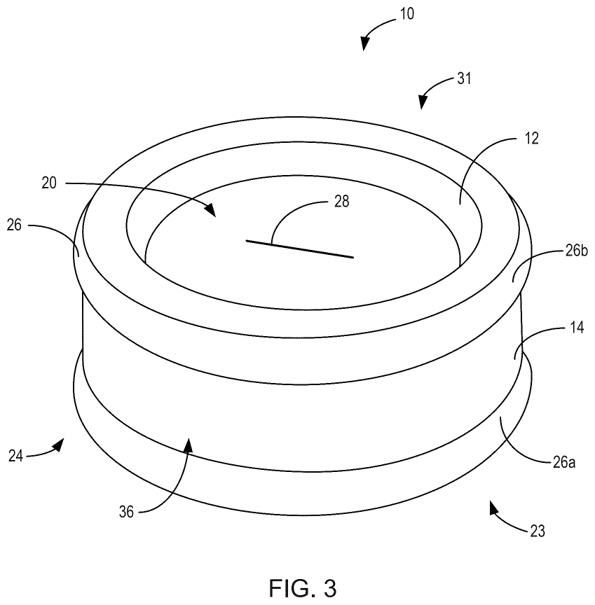

Referring now to FIGS. 3-18, shown are non-limiting embodiments of a sequential stopper 10 that may be included in injection device 100, according to aspects of the disclosure. The sequential stopper 10 generally comprises a membrane 12, and a lateral wall 14. The lateral wall 14 may extend both proximally and distally from the membrane 12 to define cavities 16, with each cavity comprising a hollow volume delimited by an inner face 18 of the lateral wall, along with a proximal face 20 and/or a distal face 22 of the membrane 12. That is, the membrane 12 may be positioned so as to be substantially centered intermediate between a distal end 23 and a proximal end 31 of the sequential stopper 10, with the lateral wall 14 extending both proximally and distally from the membrane 12 to provide two separate respective cavities 16—including a proximal cavity delimited by the inner face 18 of the lateral wall 14 and the proximal face 20 of the membrane 12 and a distal cavity delimited by the inner face 18 of the lateral wall 14 and the distal face 22 of the membrane 12. It is noted herein that the term “intermediate between a distal end and a proximal end” of the membrane 12 may include manufacturing tolerances of +/−0-20% of the overall length of the sequential stopper 10 when in an uncompressed state.

The sequential stopper 10 may be made of any material with elastomeric properties usually used to manufacture stoppers for medical injection devices. For example, the sequential stopper 10 may be made of elastomer, rubber, thermoplastic elastomer, or liquid silicon rubber. In an exemplary embodiment, the sequential stopper 10 is formed as a single, integral component (i.e., the membrane 12 and lateral wall 14 are integrally formed) and of a single material (i.e., a mono-component and mono-material stopper), with the elastomeric properties of the sequential stopper 10 allowing for flexing and/or deformation of the membrane 12 thereof. According to some non-limiting embodiments, the membrane 12 of the sequential stopper 10 may have a thickness that is between 0.2-7 mm, depending on the configuration/shape of the membrane. In certain configurations, the membrane may be flat, convex, concave, or have a multi-shape profile in which the membrane has a first portion which is at least one of flat, convex and concave, and a second portion which is another of flat convex or concave, the first portion being different from the other portion. The sizing of the sequential stopper 10 (e.g., 1, 10, or 20 mL stopper), is to provide for deflection/deformation thereof when forces are applied to the membrane 12. In the region of the membrane 12 that is collapsible or deformable (i.e., generally, a region that includes a slit 28 therein), the thickness of the membrane will preferably be from 0.2 to 3.0 mm.

As shown in FIGS. 3-18, the sequential stopper 10 has a substantially cylindrical shape, which corresponds to the shape of the barrel 106 of the injection device in which the sequential stopper 10 is intended to be inserted. Accordingly, the lateral wall 14 of the sequential stopper 10 has a generally cylindrical or tubular construction. The lateral wall 14 is provided with an outer sealing surface 24 configured to sealingly engage the inner surface 136 of the barrel 106. The sealing surface 24 is continuous, which means that it extends continuously about the circumference of the sequential stopper 10 and forms a ring. Since the continuous surface extends between the outer face of the lateral wall 14 of the sequential stopper 10 and the inner surface 136 of the barrel 106 of the injection device 100, any passage of a composition between the sequential stopper 10 and the barrel 106 is prevented. Optimal sealing is thus ensured.

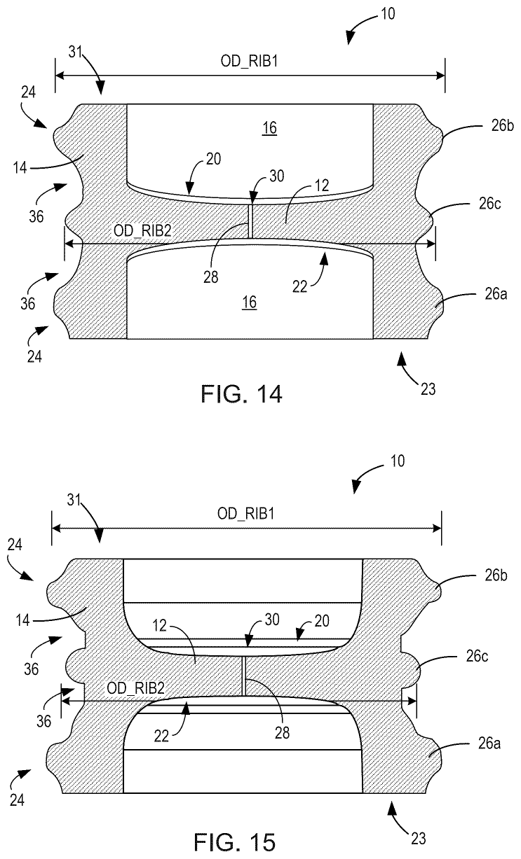

According to a preferred embodiment, the sealing surface 24 comprises a plurality of sealing ribs 26. The number of ribs 26 as well as the dimensions of each rib 26, such as height, width, and the distance between two adjacent ribs, may be adapted to as to further optimize the sealing depending on the dimensions of the sequential stopper 10 and the barrel 106. In some embodiments, such as the sequential stoppers of FIGS. 3-13, 17, and 18, the sequential stopper 10 comprises two ribs 26—i.e., a distal first rib 26a and a proximal second rib 26b. In other embodiments, such as the sequential stoppers of FIGS. 14-16, the sequential stopper 10 comprises three ribs 26—i.e., a distal first rib 26a, a proximal second rib 26b, and a center third rib 26c. Where the sequential stopper 10 includes only two ribs 26, each of the distal first rib 26a and the proximal second rib 26b is configured to provide a fluid-tight sealed interface with the syringe barrel 106, to maintain separation of fluids in the syringe 100—with the ribs 26a, 26b providing a desired amount of interference with the barrel 106. Where the sequential stopper 10 includes three ribs 26, each of the distal first rib 26a and the proximal second rib 26b is configured to provide a fluid-tight sealed interface with the syringe barrel 106, to maintain separation of fluids in the syringe 100, while the center third rib 26c may have a slightly reduced outer diameter (as compared to ribs 26a, 26b), so as to have a reduced interference with the syringe barrel 106. The center third rib 26c may thus not provide a fluid-tight seal with the barrel 106, but instead act as an anti-buckling feature that provides a radial strength to maintain the sequential stopper 10 in a closed/sealed configuration, as explained in further detail below.

The inclusion of two (or three) ribs 26 on sealing surface 24 reduces the contact surface between the lateral wall 14 of the sequential stopper 10 and the inner surface 136 of the barrel 106 of the injection device 100, compared to a plane lateral wall of the sequential stopper 10, thus improving the gliding performance of the sequential stopper 10 relative to the barrel 106. As a result, the force that needs to be exerted onto the sequential stopper 10 for displacing it inside the barrel 106 is reduced, which makes the injection easier for the user. Additionally, the inclusion of the pair of spaced apart ribs 26 on sequential stopper 10 increases the stability of the sequential stopper 10 during insertion thereof into barrel 106 (e.g., during vent tube stoppering) and during injection of the first fluid.

According to embodiments of the disclosure, the proximal face 20 and the distal face 22 of the membrane 12 may each present a flat or planar surface, a convex surface, a concave surface, or a multi-shape profile that extends or protrudes proximally or distally. The term “convex”, as relates to a face of the membrane 12, means that the face is curved, and that the apex of the curvature extends away from a plane orthogonal to the distal-proximal direction and comprising the middle of the thickness of the membrane 12 in the thinnest region thereof. Hence, the apex of the curvature of the proximal face 20 of the membrane 12, presenting a convex shape, extends away from the distal face 22 of the membrane 12. The term “concave”, as relates to a face of the membrane 12, means that the face is curved and that the apex of the curvature is directed towards a plane orthogonal to the distal-proximal direction and comprising the middle of the thickness of the membrane 12 in the thinnest region thereof. Hence, the apex of the curvature of the proximal face 20 of the membrane 12, presenting a concave shape, extends toward the distal face 22 of the membrane 12.

According to aspects of the disclosure, the membrane 12 comprises at least one slit 28 formed therein for permitting fluid flow through the membrane 12 when the slit is in an open position/configuration. The slit 28 extends through an entirety of the membrane 12, between the proximal face 20 and the distal face 22 thereof, and is configured to create a fluid path 30 through the membrane 12 from the proximal face 20 to the distal face 22 of the membrane 12 when the slit 28 is opened.

According to aspects of the disclosure, the slit 28 is configured to transition between a closed position, where fluid flow through the membrane 12 is prevented, and an open position, where fluid flow through the membrane 12 can occur, thereby establishing fluid communication between the proximal chamber 104 and the distal chamber 102 through the sequential stopper 10. In some examples, the sequential stopper 10 can be configured to remain in the closed position when a fluid pressure P₂ in the proximal chamber 104 of the syringe barrel 106 is below a predetermined opening force Fₒₚ of the slit 28—which corresponds to the force/pressure that needs to be exerted onto the proximal face 20 of the membrane 12 for opening the slit 28. The slit 28 of sequential stopper 10 can be configured to transition to the open position when the fluid pressure P₂ in the proximal chamber 104 of the syringe barrel 106 is greater than or equal to the predetermined opening force Fₒₚ of the slit 28. In some examples, the slit 28 is biased to and initially provided in the closed position (i.e., maintained closed under radial compression of the sequential stopper 10 within the barrel 106), meaning that when fluid pressure P₂ in the proximal chamber 104 is nominal or is substantially equal to fluid pressure P₁ in the distal chamber 102, the slit 28 is in the closed position. The slit 28 transitions to the open position when the fluid pressure P₂ in the proximal chamber 104 substantially increases above the opening force Fₒₚ of the slit 28.

The activation or opening force Fop for the slit 28 can be selected based on fluid pressures/forces that commonly occur when a stopper is manually moved through a barrel of a conventional syringe at a reasonable rate, as occurs when a practitioner pushes a plunger rod of a syringe through the syringe barrel. In some examples, the activation or opening force Fop for the slit 28 may be between 2 N and 25 N, preferably between 2 N and 20 N, and even more preferably between 5N and 20N. However, it is recognized that the activation or opening force Fop can be selected or optimized for different syringe designs taking into account, for example, the size, shape, and materials of the sequential stopper 10, the syringe barrel 106, and other components of the multi-chamber syringe 10. In some examples, the syringe 10 is configured such that the activation or opening pressure for the slit 28 is greater than a pressure required to infuse the drug (i.e., the pressure required to expel the first fluid from the distal chamber 102 through the tip 116 and to the patient through the needle cannula and/or through the VAD). Accordingly, the practitioner may be required to apply a greater force to a plunger rod 122 of the syringe 10 connected to the plunger stopper 124 in order to cause the slit 28 of sequential stopper 10 to open than is required to move the sequential stopper 10 (and plunger stopper 124) through the barrel 106. Due to these differences in the force that must be applied to the plunger rod 122, the practitioner can receive feedback (i.e., a feeling that increased force on the plunger rod is needed) indicating that the initial fluid has been expelled from the syringe 10 and that the sequential stopper 10 is in its distal-most position in the barrel 106.

As previously described, with sequential stopper 10 loaded into syringe barrel 106, the membrane 12 operates as a one-way membrane that functions as a check valve, which means that the membrane 12 is configured to allow a composition/fluid to pass through the membrane 12 via the open slit 28 only when the composition flows in the distal direction, i.e., the direction of injection. In other terms, the slit 28 is configured to open only for allowing the composition to pass through the membrane 12 from one side of the sequential stopper 10 where the composition contacts the proximal face 20 of the membrane 12, to another side of the sequential stopper 10 where the composition contacts the distal face 22 of the membrane 12—i.e., the second fluid F2 in proximal chamber 104 may pass through the membrane 12 into the distal chamber 102, while the first fluid F1 in distal chamber 102 may not pass through the membrane 12 into the proximal chamber. The slit 28 is configured to remain closed for preventing a composition to pass through the membrane 12 in the proximal direction—i.e., flowing from where the composition contacts the distal face 22 of the membrane 12 to where the composition contacts the proximal face 20 of the membrane 12.

According to aspects of the disclosure, the sequential stopper 10 in each of the embodiments of FIGS. 3-18 may be characterized as a “symmetrical stopper,” where the sequential stopper 10 will have an identical configuration regardless of the orientation of the sequential stopper 10 when loaded into the syringe barrel 106. That is, the sequential stopper 10 may be loaded into syringe barrel 106 in either of a first orientation or an opposite second orientation (i.e., right side up or upside down), with the sequential stopper having the same positioning and orientation of membrane 12 and slit 28 regardless of the orientation of sequential stopper 10 relative to syringe barrel 106. To provide such a symmetrical sequential stopper 10, membrane 12 is shaped on both sides—i.e., the proximal face 20 and distal face 22 to have a corresponding configuration such that the proximal face 20 and the distal face 22 are symmetrical and mirrored—and the membrane 12 is centered lengthwise between the distal end 23 and the proximal end 31. It is understood herein that the term “corresponding configuration” encompasses general expected manufacturing tolerances and that “corresponding configuration” need not be mathematically identical but would be identical in performance metrics.

A more detailed description of the various symmetrical sequential stopper configurations of FIGS. 3-18 is now provided here below.

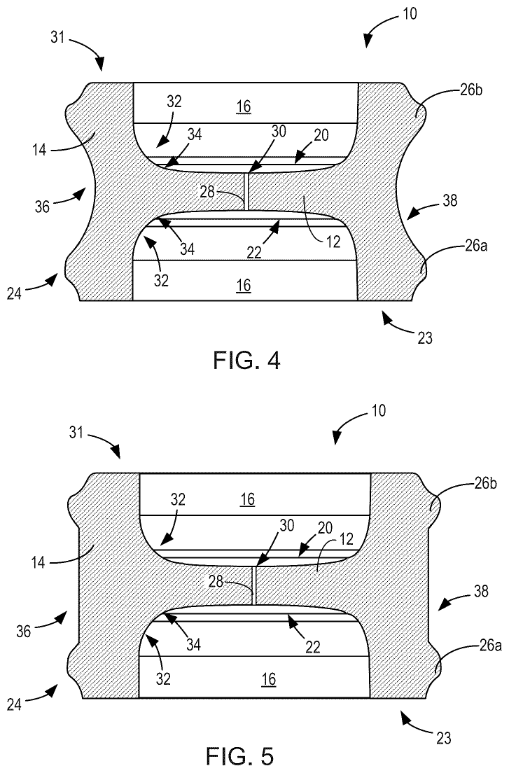

Referring to FIGS. 3 and 4, a sequential stopper 10 is illustrated that includes two ribs 26a, 26b forming a sealing surface 24—i.e., a distal first rib 26a and a proximal second rib 26b. The membrane 12 is joined with lateral wall 14 at location halfway between distal end 23 and proximal end 31, so as to be centered lengthwise on the sequential stopper 10. In one embodiment, membrane 12 may be formed to have a slightly concave distal face 22 and proximal face 20, so that the distal face 22 and proximal face 20 are mirror images of each other.

According to an embodiment of the disclosure, the membrane 12 is joined with an inner surface of the lateral wall 14 via connecting portions 32 extending between the membrane 12 and the lateral wall 14. A connecting portion 32 may be provided to join each of the distal surface 22 and the proximal surface 20 of the membrane 12 with the lateral wall 14. According to embodiments the connecting portions 32 may provide a smooth curved transition between the lateral wall 14 and the membrane 12 or may provide a sharp connection angle between the lateral wall 14 and the membrane 12. In operation of the sequential stopper 10, the connecting portions 32 may function as a hinge 34 about which portions of the membrane 12 deflect or pivot when pressure is exerted onto the proximal face 20 of the membrane 12, allowing for the slit 28 to transition to the open position—i.e., portions of the membrane 12 deflect or pivot about the hinge 34 provided by connecting portions 32 when the fluid pressure P₂ in the proximal chamber 104 substantially increases above the opening force Fₒₚ of the slit 28.

In the sequential stopper of FIGS. 3 and 4, the distal first rib 26a is separated from the proximal second rib 26b by an inter-rib region 36 of the lateral wall 14, and the inter-rib region 36 is configured as a curved inter-rib region 36. That is, in the inter-rib region 36 of the lateral wall 14, the outer surface 38 of the lateral wall 14 is curved radially inward—with a minimum outer diameter of inter-rib region 36 being located at a point equidistant between the distal first rib 26a and the proximal second rib 26b. A radius of curvature of the curved inter-rib region 36 may depend on the size/volume of the syringe 100 (e.g., 1, 10, or 20 ml syringe, as non-limiting examples), but in general the radius of curvature of the inter-rib region 36 is from 1 mm to 10 mm. Preferably, the radius of curvature of the inter-rib region 36 is from 2 mm to 3 mm.

Other variations of a two-rib sequential stopper 10 are provided in FIGS. 5-7, where each sequential stopper 10 has a similar construction to the sequential stopper 10 previously described in FIGS. 3 and 4. However, the sequential stopper 10 in each of FIGS. 5-7 differs in that the inter-rib region 36 thereof has a different configuration.

Referring to FIG. 5, a sequential stopper 10 is illustrated that includes an inter-rib region 36 between the distal first rib 26a and the proximal second rib 26b, where the inter-rib region 36 is configured as a flat region/section. The inter-rib region 36 may thus present a cylindrical outer surface 38 for the lateral wall 14.

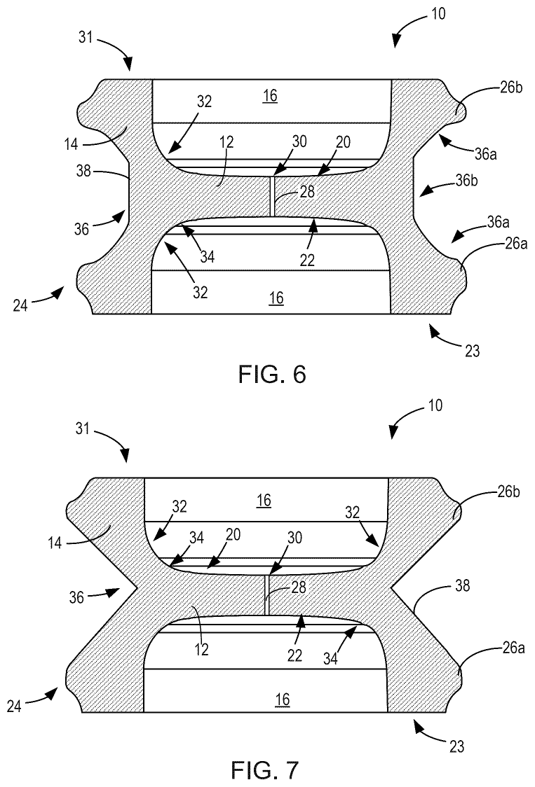

Referring to FIG. 6, a sequential stopper 10 is illustrated that includes an inter-rib region 36 between the distal first rib 26a and the proximal second rib 26b, where the inter-rib region 36 has a stepped configuration. In particular, an outer portion 36a of the inter-rib region 36 (adjacent to each of ribs 26a, 26b) has a curved or sloped profile that is oriented radially inward, while a central portion 36b of the inter-rib region 36 (between the outer portions) is configured as a flat region/section.

Referring to FIG. 7, a sequential stopper 10 is illustrated that includes an inter-rib region 36 between the distal first rib 26a and the proximal second rib 26b, where the inter-rib region 36 has a V-shaped configuration. In particular, the outer surface 38 of the lateral wall 14 is sloped radially inward from each of the ribs 26, 26b, to meet at a center point of the inter-rib region 36.

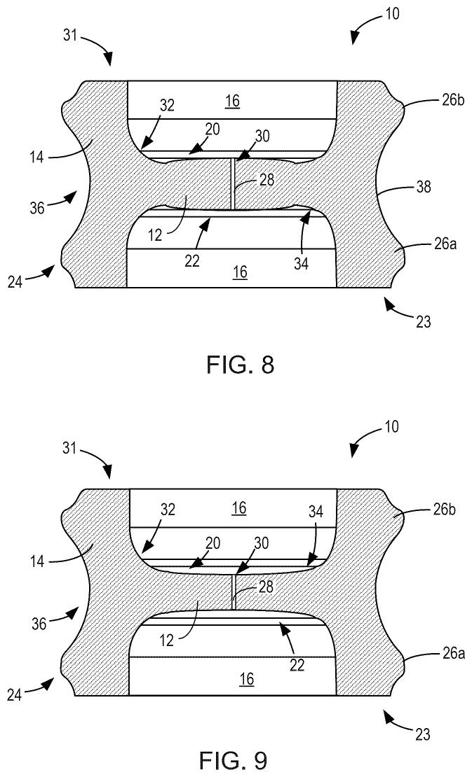

For each of the embodiments of FIGS. 3-7, it is recognized that the shape or profile of the proximal face 20 and the distal face 22 of the membrane 12 may differ from those shown—i.e., a shape/profile other than the concave profile of FIGS. 3-7. That is, as previously described, the proximal face 20 and the distal face 22 of the membrane 12 may each present a flat or planar surface, a convex surface, a concave surface, or multi-shape profile that extends or protrudes proximally or distally. Thus, as additional examples, the sequential stopper 10 is shown in FIGS. 8 and 9 with membrane 12 configured such that the proximal face 20 and the distal face 22 have a convex shape/profile (FIG. 8) or a flat shape/profile (FIG. 9). While FIGS. 8 and 9 show a sequential stopper 10 similar to FIGS. 3 and 4 where the inter-rib region 36 is configured as a curved inter-rib region 36, it is recognized that a membrane 12 having a proximal face 20 and distal face 22 with a convex shape/profile or a flat shape/profile could also be incorporated into the sequential stopper of FIG. 5, FIG. 6, or FIG. 7.

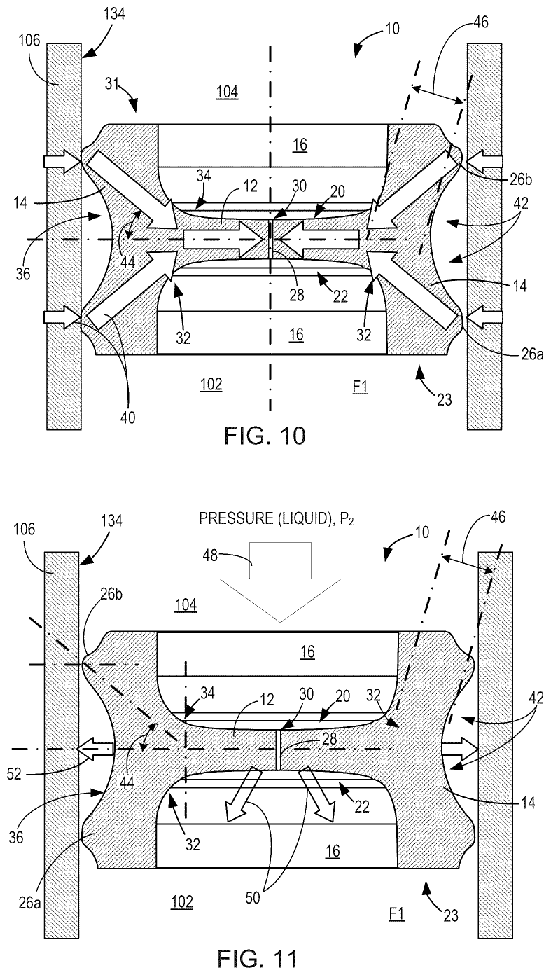

Referring now to FIGS. 10 and 11, diagrams illustrating forces applied on the membrane 12 of sequential stopper 10 during use of the syringe 100 are provided. While the sequential stopper 10 illustrated in FIGS. 10 and 11 are identical to the sequential stopper 10 shown in FIGS. 3 and 4, it is recognized that similar forces would be present on any of the sequential stopper of FIGS. 5-9. FIG. 10 illustrates compressive forces that are transferred to the membrane 12 resulting from placement of the sequential stopper 10 within syringe barrel 106 and the interference present between the sequential stopper 10 and syringe barrel 106. FIG. 11 illustrates the fluid pressure P₂ in the proximal chamber 104 that is applied against the proximal face 20 of the membrane 12 during an injection sequence for the syringe 100, along with resulting movement/deflection of the membrane 12 and lateral wall 14 that occurs upon the fluid pressure P₂ causing the slit 28 to open (i.e., when the fluid pressure P₂ is greater than the opening force Fop of the slit 28).

Referring first to FIG. 10, the compressive forces acting on membrane 12 are illustrated generally by arrows 40. The compressive forces that are transferred to membrane 12 may be present in what is termed herein as a “force transfer area” 42 that comprises a lengthwise region that extends between the proximal rib 26b and the membrane 12 and between the distal rib 26a and the membrane 12. In this force transfer area 42, a radially inward-directed compressive force from the syringe barrel 106 pressing against the ribs 26a, 26b is transferred to the membrane 12 (through lateral wall 14 and connecting portions 32)—with the radially inward-directed compressive force aiding in maintaining the slit 28 in a closed position and preventing passage of fluid by fluid path 30 as fluid path 30 is completely sealed.

As shown in FIG. 10, the radially inward-directed compressive force that is transferred to membrane 12 (via the lateral wall 14 and connecting portions 32) may be directed along an angle of force transfer from the ribs 26a, 26b to the membrane 12, as indicated at 44. The angle of force transfer 44 may be defined along a line extending from a rib contact axis to a hinge point 34 of a connecting portion 32. According to embodiments, it is desirable for the angle of force transfer 44 to be between 20 degrees and 60 degrees, and preferably between 30 degrees and 45 degrees, in order for the compressive force to be adequately transferred to membrane 12 to maintain/bias the slit 28 in its closed position until a fluid pressure P₂ greater than the opening force Fop of the slit 28 is applied to the membrane 12. In particular, with the angle of force transfer 44 provided in this range, the compressive force transferred to membrane 12 aids in maintaining/biasing the slit 28 in its closed position during injection of a first fluid F1 from the distal chamber 102.

According to embodiments, and in order to provide for transferring of the radially inward-directed compressive force to membrane 12, a thickness of the lateral wall 14 and connecting portions 32 in the force transfer area 42 may be set to a desired thickness. The lateral wall 14 and connecting portions 32 in the force transfer area 42 should have a minimum thickness, indicated at 46, that provides sufficient structural integrity to the sequential stopper 10 and provides for a transferring of the radially inward-directed compressive force to membrane 12 and should have a maximum thickness that provides for suitable deflection of the lateral wall and connecting portions 32 during opening of the slit 28. According to embodiments, it is desirable for a thickness 46 of the lateral wall 14 and connecting portions 32 in the force transfer area 42 to be between 0.5 mm and 2 mm.

Referring now to FIG. 11, application of a fluid pressure P₂ in the proximal chamber 104 against the proximal face 20 of the membrane 12 during an injection sequence for the syringe 100 is shown, with the pressure illustrated generally by arrow 48. When the fluid pressure P₂ applied to the proximal face 20 of the membrane 12 is greater than the opening force Fop of the slit 28, the membrane 12 is caused to deflect at a location of the hinge 34 of connecting portions 32, such that slit 28 is caused to open (as indicated by arrows 50) and provide a fluid path 30 through the membrane 12 for transmission of a fluid therethrough in the distal direction. In addition to the deflection of membrane 12, the lateral wall 14 of sequential stopper 10 is also caused to deflect, as indicated by arrows 52, to allow for wider opening of the slit 28 to allow opening of the fluid path 30. Specifically, the lateral wall 14 deflects radially outward in the area of inter-rib region 36.

With regard to the sequential stopper 10 illustrated in FIGS. 10 and 11, it is recognized that the configuring of lateral wall 14 to include a curved inter-rib region 36 (or, similarly, a stepped or V-shaped inter-rib region 36, as shown in FIGS. 6 and 7) and the controlling of the thickness 46 of the lateral wall 14 and connecting portions 32 in the force transfer area 42 provide for a desirable transfer of forces in sequential stopper 10 and for a desirable deflection of the sequential stopper 10 during operation. Specifically, the curvature of inter-rib region 36 and the thickness 46 of the lateral wall 14 and connecting portions 32 in the force transfer area 42 (along with a desired angle of force transfer 44), (1) enables an effective transfer of a radially inward-directed compressive force to membrane 12 (along the angle of force transfer 44) to maintain/bias the slit 28 in its closed position, and (2) allows for an outward deflection of the inter-rib region 36 during opening of slit 28, without the lateral wall 14 in inter-rib region 36 contacting the syringe barrel 12 (so as to keep a gliding force lower for sequential stopper 10). The curvature of inter-rib region 36 also functions to enhance the stability of the sequential stopper 10 during ageing and reduce operational variability (e.g., the opening force Fop of the slit 28 may be kept stable/constant after an ageing period of 5 years, as a non-limiting example).

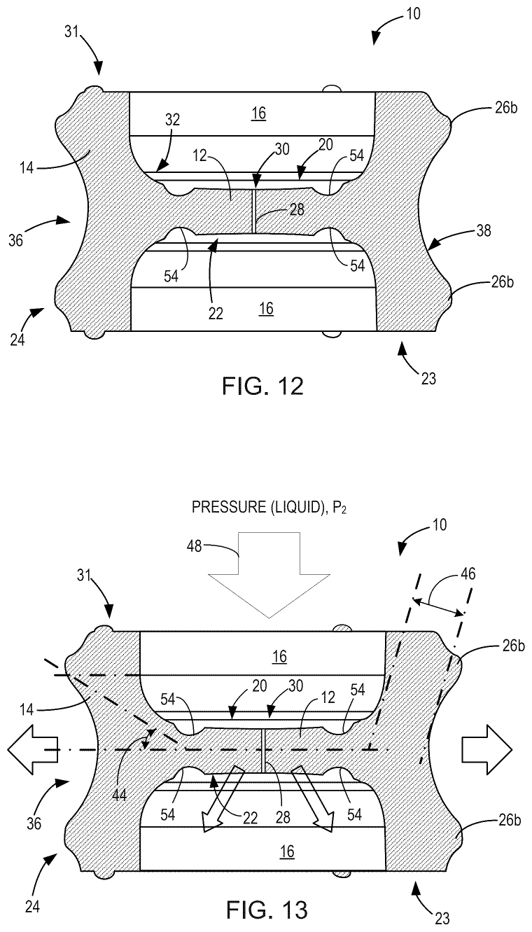

Referring now to FIGS. 12 and 13, a sequential stopper 10 is shown according to another embodiment of the disclosure. The sequential stopper 10 may have a similar construction to the sequential stopper previously described in FIGS. 3 and 4. However, the sequential stopper 10 in each FIGS. 12 and 13 differs in that the membrane 12 further includes a plurality of trenches or gutters 54 formed therein. The gutters 54 may be formed on the proximal face 20 of the membrane 12, the distal face 22 of the membrane 12, or on both the proximal face 20 and the distal face 22 of the membrane 12. The gutters 54 extend across a portion of the proximal and/or distal faces 20, 22 and may comprise one of a linear segment, a curved segment, or a mixture of a linear segment and a curved segment.

The gutters 54 reduce the thickness of the membrane 12 at the location(s) thereof, such that the gutters 54 function as hinges about which portions of the membrane 12 may deflect when transitioning from its closed position to its open position. The configuration of each gutter 54, including the shape and/or depth thereof, may be selected or tuned in order to adjust the opening force Fop of the corresponding slit 28, as well as a speed at which the slit 28 opens. In some embodiments, the depth of the gutters 34 is such that the remaining thickness of the membrane 12 at the locations thereof is 20% to 70% of the overall thickness of the membrane 12, with the hinge thickness at the gutters 34 impacting the opening force Fop of the corresponding slit 28.

FIG. 13 illustrates the fluid pressure P₂ in the proximal chamber 104 that is applied against the proximal face 20 of the membrane 12 during an injection sequence for the syringe 100, along with resulting movement/deflection of the membrane 12 and lateral wall 14 that occurs upon the fluid pressure P₂ causing the slit 28 to open (i.e., when the fluid pressure P₂ is greater than the opening force Fₒₚ of the slit 28). As shown in FIG. 13, an angle of force transfer 44 in the sequential stopper 10 is defined along a line extending from a rib contact axis to a hinge point at the centerline of the gutter(s).

Referring now to FIGS. 14 and 15, a sequential stopper 10 is illustrated that includes three ribs 26a, 26b, 26c—i.e., a distal first rib 26a, a proximal second rib 26b, and a center third rib 26c. The membrane 12 is joined with lateral wall 14 at location halfway between distal end 23 and proximal end 31, so as to be centered lengthwise on the sequential stopper 10, and the membrane 12 is also aligned lengthwise with the center third rib 26c. In one embodiment, membrane 12 may be formed to have a slightly concave distal face 22 and proximal face 20, so that the distal face 22 and proximal face are mirror images of each other. In other embodiments, membrane 12 may be formed to have a distal face 22 and proximal face 20 that are each convex or flat in shape, as shown in FIGS. 8 and 9.

In the sequential stopper of each of FIGS. 14 and 15, an inter-rib region 36 of the lateral wall 14 is provided between the distal first rib 26a and the center third rib 26c and between the proximal second rib 26b and the center third rib 26c. According to embodiments, each inter-rib region 36 may be configured as a curved inter-rib region 36 (FIG. 14) or a flat inter-rib region 36 (FIG. 15).

According to embodiments of the disclosure, the center third rib 26c is constructed to have a reduced diameter as compared to the distal first rib 26a and proximal second rib 26b. That is, the distal first rib 26a and proximal second rib 26b are formed to have a first outer diameter, OD_rib1, and the center third rib is formed to have a second outer diameter, OD_rib2, that is smaller than the first outer diameter. With the center third rib formed to have a second outer diameter, OD_rib2, the center third rib 26c is not configured to form part of an outer sealing surface 24 configured to sealingly engage the inner surface 136 of the barrel 106 (FIG. 1), but instead the center third rib 26c is configured to aid in maintain the slit 28 in a closed position, such as during injection of a first fluid F1 from the distal chamber 102 of syringe barrel 106.

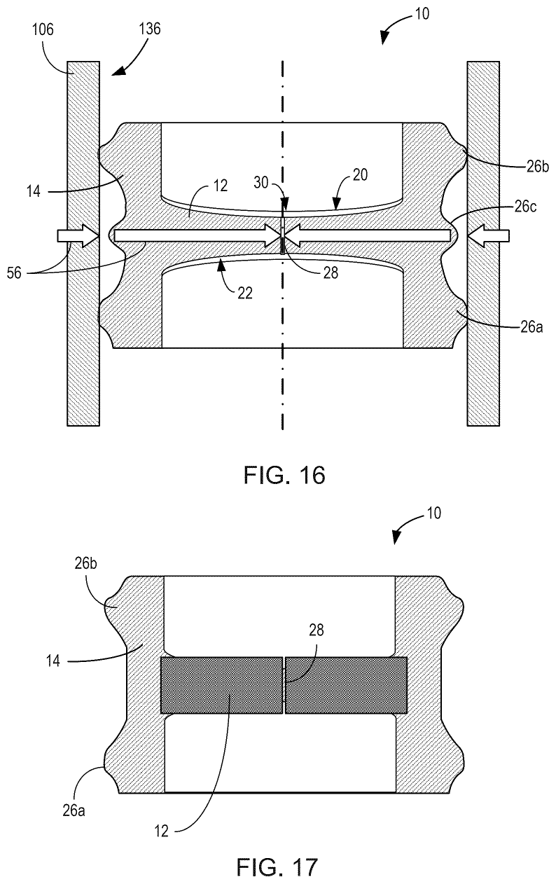

Referring now to FIG. 16, a diagram illustrating compressive forces that are transferred to the membrane 12 resulting from placement of the sequential stopper 10 within syringe barrel 106 and the interference present between the sequential stopper 10 and syringe barrel 106 is provided. In FIG. 16, the compressive forces acting on membrane 12 are illustrated generally by arrows 56—with it being shown therein that a radially inward-directed compressive force from the syringe barrel 106 pressing against center third rib 26c is transferred to the membrane 12 through the center third rib 26c—with the radially inward-directed compressive force aiding in maintaining the slit 28 in a closed position. As indicated above, a diameter of the center third rib 26c, OD_rib2, is less than a first outer diameter, OD_rib1, of the distal first rib 26a and proximal second rib 26b, with the center third rib 26c sized large enough to provide for such compressive force to be applied thereto, but not large enough to add substantially to the gliding force required to move sequential stopper 10 within syringe barrel 106—i.e., there is a smaller amount of interference between the center third rib 26c and the syringe barrel 106, as compared to ribs 26a, 26b.

For any of the embodiments of sequential stopper 10 shown in FIGS. 3-16, it is recognized that the sequential stopper 10 may be formed as a mono-material valve or a bi-material valve.

For a mono-material sequential stopper, such as shown in FIGS. 3-16, the sequential stopper 10 is formed as a single, integral component (i.e., the membrane 12 and lateral wall 14 are integrally formed) and of a single material (i.e., a mono-component and mono-material stopper), with the elastomeric properties of the sequential stopper 10 allowing for flexing and/or deformation of the membrane 12 thereof. For example, the sequential stopper 10 may be made of elastomer, rubber, thermoplastic elastomer, or liquid silicon rubber, as non-limiting examples.

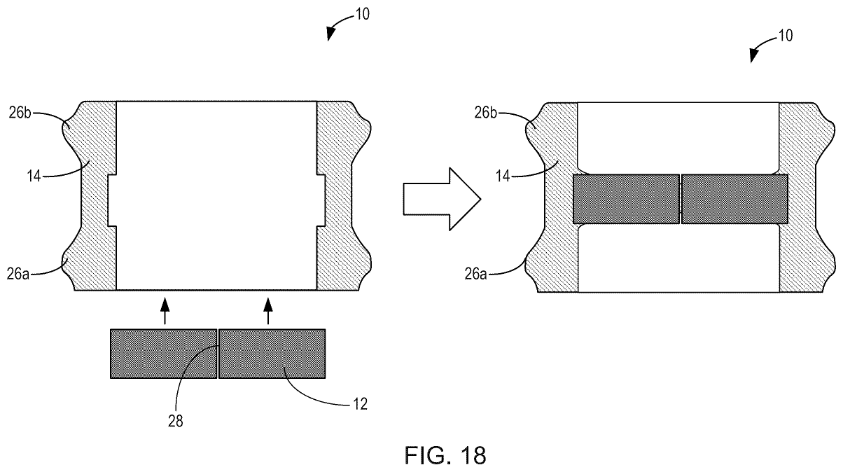

For a bi-material sequential stopper, such as shown in FIGS. 17 and 18, the sequential stopper 10 is formed as of two separate components, each formed of a different material. Specifically, the lateral wall 14 (and ribs 26 thereon) may be formed as a first component and from a first material, while the membrane 12 may be formed as a second component and from a second material. In some embodiments, the lateral wall 14 may be formed of rubber, while the membrane 12 may be formed of liquid silicon rubber. The bi-material sequential stopper 10 may be formed via a number of suitable manufacturing techniques, such as a two-step overmolding process (FIG. 17) where the lateral wall 14 is overmolded onto the membrane 12 or as a two-step assembly process (FIG. 18) where a pre-formed membrane 12 is inserted/placed into a pre-formed lateral wall 14 (e.g., into a notch 58 in lateral wall 14).

As previously described, the injection device 100 enables the sequential delivery of an initial fluid F1 and a second fluid F2 through a single continuous advancement of a plunger rod 122 of the injection device 100. The first fluid F1 and second fluid may have similar or dissimilar properties, including a concentration, viscosity, and/or pressure of the fluids, as non-limiting examples. The type and amount of solution contained in the proximal chamber 104 and/or the distal chamber 102 may vary depending, for example, on the specific configuration of the injection device 100 and/or on the therapeutic effect to be achieved. In some examples, the injection device 100 contains or is configured to contain between about 0.1 mL and 20 mL of the initial fluid F1 and/or the secondary fluid F2. Additionally, the structure of the sequential stopper 10—in particular membrane 12—may be specifically configured based on properties of the fluids F1, F2 contained within the syringe. That is, the pressure and/or viscosity of the fluids F1, F2 may dictate the thickness of the membrane 12 and/or a configuration and sizing of the slit 28.

According to aspects of the disclosure, the first fluid F1 and the second fluid F2 may—in general—comprise a liquid-liquid combination of pharmaceuticals in one of a number of drug classes or categories designed to treat a recognized condition. Such drug classes or categories may include analgesics, vitamins, vaccines (and boosters), monoclonal antibodies, diabetes and obesity treatments, and the like.

One exemplary embodiment of a liquid-liquid combination of a first fluid F1 and second fluid F2 that may be sequentially injected from the first chamber 102 and second chamber 104 comprises a fixed-dose combination of diabetes/obesity drugs—where the first fluid F1 and the second fluid F2 contained within the distal chamber 102 and the proximal chamber 104 of injection device may comprise a GLP-1 agonist and an amyline analog, respectively. The GLP-1 agonist may be a calcitonin receptor agonist, including any of Dulaglutide, Exenatide, Semaglutide, or Liraglutide, as non-limiting examples. The amyline analog may be dual amylin, including any of Cagrilintide or pramlintide, as non-limiting examples.

In one exemplary embodiment, the injection device 100 enables a delivery/injection of a two drug combination to a patient, such as a first fluid F1 comprising cagrilintide and a second fluid F2 comprising semaglutide, and with the cagrilintide and semaglutide being sequentially delivered/injected through a single continuous advancement of the plunger rod 122 of the injection device 100.

Injection of a medication/treatment such as, for example, a GLP-1 agonist and an amyline analog which are sequentially injected (via injection device 100 in a non-limiting example), and it is recognized that other liquid-liquid combinations of a first fluid F1 and second fluid F2 may be sequentially injected from the first chamber 102 and second chamber 104 of the injection device 100. As indicated above, injection device 100 may also be utilized for sequential injections of other liquid-liquid combination of pharmaceuticals, including analgesics, vitamins, vaccines (and boosters), monoclonal antibodies, and the like.

FIG. 19A corresponds to the configuration of the injection device 100 before injection of the first fluid F1. In this configuration, the plunger stopper 124 is in a proximal position and the sequential stopper 10 is in a rest position, with the slit 28 being maintained closed under radial compression of the sequential stopper, such that the fluid path 30 is closed. That is, when the sequential stopper 10 is inserted in the barrel 106 of the injection device 100, the slit 28 is maintained closed under radial compression of the sequential stopper 10, the sequential stopper 10 being itself subjected to radial compression of the barrel 106. With the plunger stopper 124 in the proximal position, the pressure P₁ in the distal chamber 102 and the pressure P₂ in the proximal chamber 104 are substantially equal, such that the differential pressure ΔP=P₂−P₁ is substantially null, and thus much lower than the valve opening force Fₒₚ of the slit 28, so that the slit 28 thus remains closed. The closed slit 28 prevents the mixing of the first fluid F1 and second fluid F2, by preventing the first fluid F1 from entering the proximal chamber 104 and the second fluid F2 from entering the distal chamber 102. Additionally, the ribs 26 of the sequential stopper 10 sealingly engage the inner surface 136 of the barrel 106, so that the first and the second fluids cannot pass from a chamber to another via a passage between the sequential stopper 10 and the barrel 106.

Referring now to FIG. 19B, the injection device 100 is shown being actuated by a user to perform the injection of the first fluid. The force applied to the plunger stopper 124 is transmitted to the second fluid F2 in the proximal chamber 104 and then to the sequential stopper 10, which results in a pressure P₂ exerted by the second fluid onto the proximal face 20 of the membrane 12 of the sequential stopper 10—which causes the sequential stopper 10 to be advanced/displaced distally within barrel 106. The displacement of the sequential stopper 10 in the distal direction pushes the first/initial fluid F1 in the distal direction, so that the first/initial fluid is expelled from the injection device 100 through the channel 118 of tip 116.

In the configuration of FIG. 19B, the sequential stopper 10 is in a sealing position, with the slit 28 remaining closed. That is, the displacement of the sequential stopper 10 builds up pressure in the distal chamber 102, since the diameter of the channel 118 is much smaller than the diameter of the barrel 106. This increase of the pressure P₁ not only forces the first fluid F1 through the channel 118 for injection, but also applies force onto the distal face 22 of the membrane 12 of the sequential stopper 10 in opposition to the displacement of the sequential stopper 10. As a result, the sequential stopper 10 is subjected to substantially equal and opposite pressures P₁ and P₂ respectively exerted by the first fluid F1 and the second fluid F2 onto the proximal face 20 and the distal face 22 of the membrane 12. As a consequence, the differential pressure ΔP is substantially null, and thus much lower than the valve opening force Fₒₚ required to open the slit 28. During the displacement of the sequential stopper 10, the differential pressure ΔP may not be substantially null but remains lower than the valve opening force Fₒₚ of the slit 28. The slit 28 thus remains closed. The injection continues until the sequential stopper 10 abuts the end wall 114 at the distal end 108 of the barrel 106, as illustrated in FIG. 19B.

Referring now to FIG. 19C, the user continues to apply a distally directed force to the plunger stopper 124. Since the sequential stopper 10 cannot move further distally, the pressure P₂ in the proximal chamber 104 increases as the plunger stopper 124 continues to advance distally, and thus the force differential ΔP becomes superior to the valve opening force Fₒₚ of slit 28. As a result, the slit 28 in membrane 12 is caused to open, thereby allowing the second fluid F2 to pass through the slit 28 and to/through the channel 118 in tip 116 for the injection. In this configuration, the sequential stopper 10 is in an injection position.

As shown in FIG. 19D, at the end of the injection of the second fluid F2, the plunger stopper 124 abuts the sequential stopper 10. At this stage, a fraction of the second fluid may remain in a “dead volume” of the proximal chamber 104—i.e., within the cavity 16 of sequential stopper 10, which is open to the proximal chamber 104. This fraction of the second fluid F2 may be forced out from the proximal chamber 104 by collapsing the sequential stopper 10 (i.e., collapsing the lateral wall 14) under the pressure exerted by the plunger stopper 124, which reduces the volume of the cavities 16, and the dead volume is thus injected.

As shown in the embodiment of FIGS. 19A-19D, the sequential stopper 10 is configured as a symmetrical stopper—where the membrane 12 has a corresponding configuration—i.e., the proximal face 20 and distal face 22 are symmetrical and mirrored within manufacturing tolerances—and the membrane 12 is centered lengthwise between the distal end 23 and the proximal end 31 of the stopper. With such a construction, the sequential stopper 10 may be loaded into syringe barrel 106 in either of a first orientation or an opposite second orientation (i.e., right side up or upside down), with the sequential stopper having the same positioning and orientation of membrane 12 and slit 28 regardless of the orientation of sequential stopper 10 relative to syringe barrel 106. Accordingly, any risk of inserting the stopper 10 into the barrel 106 (e.g., via a vent tube stoppering process) is eliminated.

While embodiments of the disclosure described above are directed to use of a sequential stopper 10 in a multi-chamber injection device 100 for performing of a sequential injection of a first fluid and a second fluid therefrom, it is recognized that sequential stopper 10 may also be used in an injection device 100 for the injection of a single fluid therefrom.

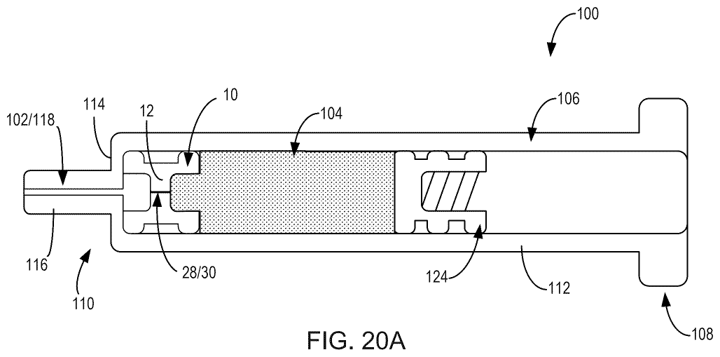

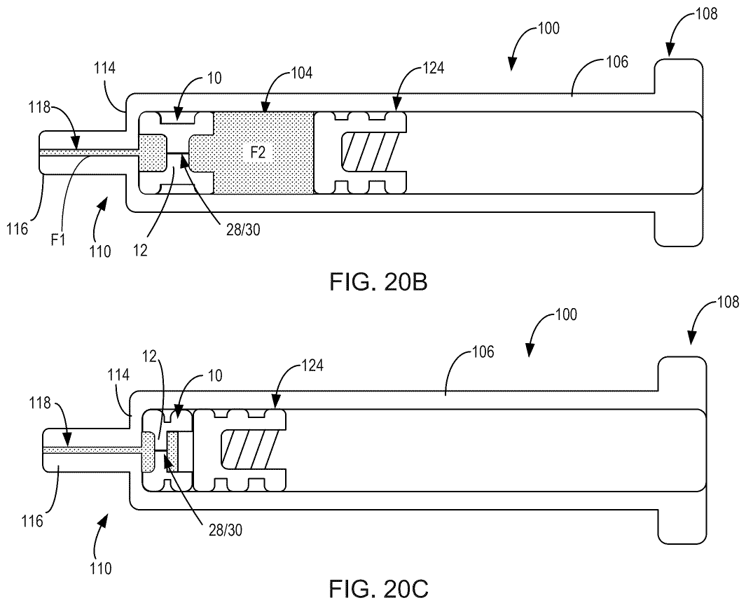

Referring now to FIG. 20A, inclusion of a sequential stopper—such as any of the sequential stoppers 10 of FIGS. 3-18—in an injection device 100 for injecting a single fluid is illustrated, in accordance with another embodiment of the disclosure. The structure of injection device 100 may be identical to that previously described in FIGS. 19A-19C regarding barrel 106 (including end wall 114 and tip 116) and plunger stopper 124; however, the sequential stopper 10 is initially positioned at the far distal end 108 of the barrel 106, so as to abut the end wall 114. In this position, the sequential stopper 10 separates two “chambers” of the barrel 106, including a distal chamber 102 defined as the channel 118 within tip 116, and a proximal chamber 104 between the sequential stopper 10 and the plunger stopper 124. In some embodiments, the distal chamber 102 (i.e., channel 118) may be empty, i.e., the “first fluid” in the distal chamber 102 is air, while the proximal chamber 104 contains a single pharmaceutical fluid. As shown in FIG. 20A, the membrane 12 of the sequential stopper 10 will be proximally offset from the end wall 114 of barrel 106, so as to allow for deflection/deformation of the membrane 12 for purposes of opening the slit 28 therein.

The functioning of the sequential stopper 10 and the injection device comprising the sequential stopper 10 will now be described herebelow, in reference to FIGS. 20A to 20C.

FIG. 20A corresponds to the configuration of the injection device 100 before injection of the single fluid. In this configuration, the plunger stopper 124 is in a proximal position and the sequential stopper 10 is in a rest position, with the slit 28 being maintained closed under radial compression of the sequential stopper 10, such that the fluid path 30 is closed. That is, when the sequential stopper 10 is inserted in the barrel 106 of the injection device 100, the slit 28 is maintained closed under radial compression of the sequential stopper 10, the sequential stopper 10 being itself subjected to radial compression of the barrel 106. With the plunger stopper 124 in the proximal position, the pressure P₂ in the in the proximal chamber 104 is much lower than the valve opening force Fop of the split, so that the slit 28 thus remains closed. The closed slit 28 prevents the single fluid from leaking into channel 118 of tip 116.

Referring now to FIG. 20B, the injection device 100 is shown being actuated by a user to perform the injection of the single fluid. The force applied to the plunger stopper 124 causes the pressure P₂ in the proximal chamber 104 to increase as the plunger stopper 124 continues to advance distally, which results in a pressure P₂ being applied by the single fluid onto the proximal face 20 of the membrane 12 of the sequential stopper 10 that becomes superior to the valve opening force Fop. As a result, the slit 28 in membrane 12 is caused to open, thereby allowing the single fluid to pass through the slit 28 and to/through the channel 118 in tip 116 for the injection. In this configuration, the sequential stopper 10 is in an injection position.

As shown in FIG. 20C, at the end of the injection of the single fluid, the plunger stopper 124 abuts the sequential stopper 10. At this stage, a fraction of the single fluid may remain in a “dead volume” of the proximal chamber 104—i.e., within the cavity 16 of sequential stopper 10. This fraction of the single fluid may be forced out from the proximal chamber 104 by collapsing the sequential stopper 10 (i.e., collapsing the lateral wall 14) under the pressure exerted by the plunger stopper 124, which reduces the volume of the cavity 16, and the dead volume is thus injected.

While embodiments of the injection device 100 described above include only a single sequential stopper 10 therein, it is recognized that other embodiments of injection devices may include one or more additional sequential stoppers 10 therein—such that a multi-chamber injection device may be used to sequentially inject a plurality of different fluids.

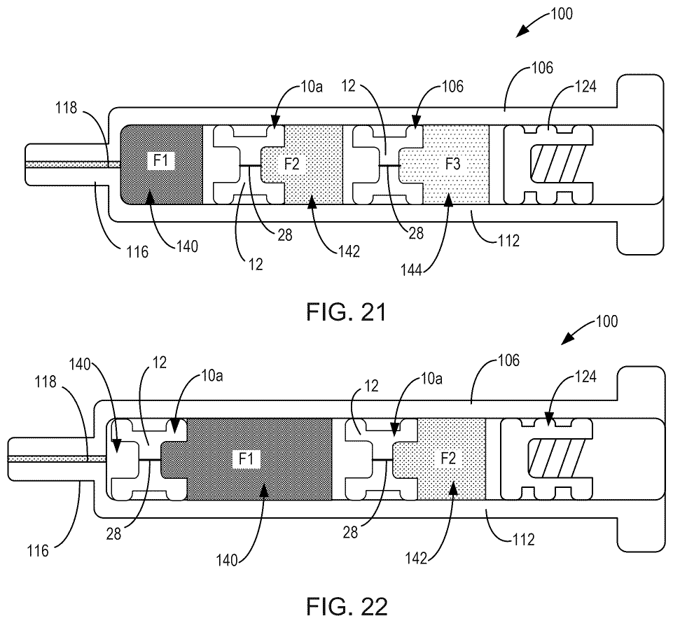

Referring now to FIGS. 21 and 22, embodiments of a multi-chamber injection device 100 are shown that include two separate sequential stoppers 10a, 10b therein for separating a plurality of fluids and allow for sequential injection of those fluids.

In FIG. 21, the multi-chamber injection device 100 includes a first sequential stopper 10a and a second sequential stopper 10b that—along with plunger stopper 124 and barrel 106—define a first chamber 140 that contains a first fluid F1, a second chamber 142 that contains a second fluid F2, and a third chamber 144 that contains a third fluid F3.

In an initial configuration before injection of the first fluid F1, the plunger stopper 124 is in a proximal position and the sequential stoppers 10a, 10b are in a rest position, with the slit 28 in each sequential stopper 10a, 10b being maintained closed under radial compression thereof.

The injection device 100 may then be actuated by a user to perform the injection of the first fluid F1—with a distally directed force applied to the plunger stopper 124. The force applied to the plunger stopper 124 is transmitted to the sequential stoppers 10a, 10b via pressure from the second and third fluids F2, F3, which causes the stoppers 10a, 10b to be advanced/displaced distally within barrel 106. The displacement of the stoppers 10a, 10b in the distal direction pushes the first fluid F1 in the distal direction, so that the first fluid F1 is expelled from the injection device 100 through the channel 118 of tip 116. During expulsion of the first fluid F1, the stoppers 10a, 10b remain in a sealing position—with the slit 28 in each sequential stopper remaining closed. The injection of the first fluid F1 continues until the sequential stopper 10a abuts the end wall 114 at the distal end 108 of the barrel 106.

The injection device 100 may then be actuated by a user to perform the injection of the second fluid F2—with a distally directed force applied to the plunger stopper 124. Since the sequential stopper 10a cannot move further distally, the pressure in the second chamber 142 increases as the plunger stopper 124 continues to advance distally, and thus the force differential ΔP becomes superior to the valve opening force Fop of slit 28 in the membrane 12 of first sequential stopper 10a. As a result, the slit 28 in membrane 12 is caused to open, thereby allowing the second fluid F2 to pass through the slit 28 and to/through the channel 118 in tip 116 for the injection. At the same time that the second fluid F2 is flowing through slit 28 of sequential stopper 10a, the sequential stopper 10b is displaced in the distal direction. During expulsion of the second fluid F2, the stopper 10b remains in a sealing position. The injection of the second fluid F2 continues until the sequential stopper 10b abuts the sequential stopper 10a.

As the user continues to apply a further distally directed force to the plunger stopper 124, pressure in the third chamber 144 increases as the plunger stopper 124 continues to advance distally, and thus the force differential ΔP becomes superior to the valve opening force Fₒₚ of slit 28 in both the sequential stoppers 10a, 10b. As a result, the slit 28 in the membrane 12 of each sequential stopper 10a, 10b is caused to open, thereby allowing the third fluid F3 to pass through the slits 28 and to/through the channel 118 in tip 116 for the injection.

In FIG. 22, the multi-chamber injection device 100 includes a first sequential stopper 10a and a second sequential stopper 10b that—along with plunger stopper 124 and barrel 106—define a first chamber 140 that remains empty, a second chamber 142 that contains a first fluid F1, and a third chamber 144 that contains a second fluid F2. In this embodiment, sequential stopper 10a is initially positioned at the far distal end 108 of the barrel 106, such that first chamber 140 is defined as the channel 118 within tip 116.

Operation of the injection device 100 in FIG. 22 may proceed similarly to as previously described for the injection devices of FIGS. 19A-19D, 20A-20C and 21, and thus is not described in detail herein.

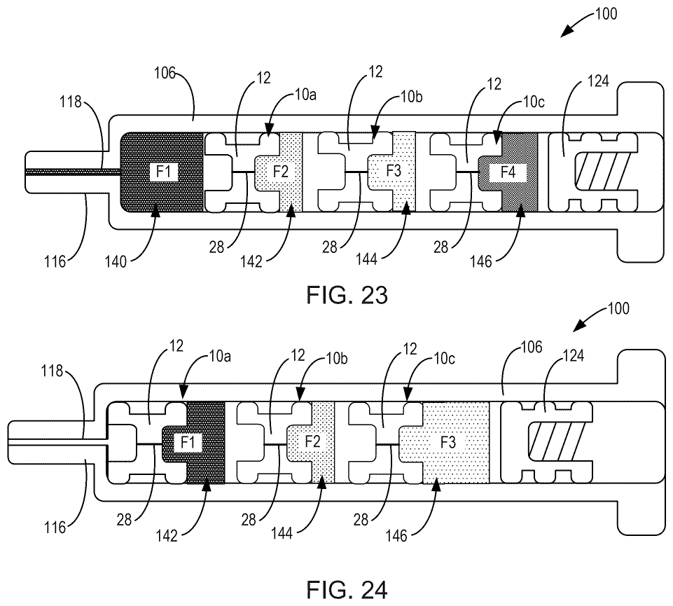

Referring now to FIGS. 23 and 24, embodiments of a multi-chamber injection device 100 are shown that include three separate sequential stoppers 10a, 10b, 10c therein for separating a plurality of fluids and allow for sequential injection of those fluids.

In FIG. 23, the multi-chamber injection device 100 includes a first sequential stopper 10a, a second sequential stopper 10b, and a third sequential stopper 10c that—along with plunger stopper 124 and barrel 106—define a first chamber 140 that contains a first fluid F1, a second chamber 142 that contains a second fluid F2, a third chamber 144 that contains a third fluid F3, and a fourth chamber 146 that contains a fourth fluid F4.

Operation of the injection device 100 in FIG. 23 may proceed similarly to as previously described for the injection device of FIG. 21, except that an additional round of sequential stopper advancement and opening is included, due to the additional sequential stopper and fluid provided.

In FIG. 24, the multi-chamber injection device 100 includes a first sequential stopper 10a, a second sequential stopper 10b, and a third sequential stopper 10c that—along with plunger stopper 124 and barrel 106—define a first chamber 140 that remains empty, a second chamber 142 that contains a first fluid F1, and a third chamber 144 that contains a second fluid F2, and a fourth chamber 146 that contains a third fluid F3.

Operation of the injection device 100 in FIG. 24 may proceed similarly to any of the previously described injection devices of FIGS. 19-23, and thus is not described in detail herein.

Beneficially, embodiments of the invention thus are directed to a sequential stopper having a symmetrical construction. A membrane of the stopper is positioned intermediate between a distal end and a proximal end thereof and includes a proximal face and a distal face having a corresponding configuration. The sequential stopper is designed to ensure proper sealing thereof with the barrel and a proper sealing of a slit or fluid path through the membrane, to maintain a separation of first and second fluids prior to injection of the second fluid. The sequential stopper also is designed to provide for opening of the slit by a desired amount, to enable an efficient fluid flow through the membrane during injection of the second fluid.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A sequential stopper configured to be positioned inside a barrel of a multi-chamber injection device for injecting at least one fluid through a distal end of the barrel, the sequential stopper comprising:
a membrane comprising a proximal face and a distal face, the membrane being configured to separate a first, distal chamber of the barrel from a second, proximal chamber of the barrel, with the membrane including at least a slit extending therethrough between the proximal face and the distal face, with the slit configured to selectively create a fluid path through the membrane from the proximal face to the distal face depending on the pressure exerted by a composition onto the proximal face of the membrane for transferring fluid only from the proximal chamber to the distal chamber; and
a lateral wall joined to the membrane to define a distal cavity and a proximal cavity, the lateral wall comprising a sealing surface including a plurality of circumferential ribs configured to sealingly engage the inner surface of the barrel;
wherein the sequential stopper comprises a symmetrical stopper where the proximal face and the distal face have a corresponding configuration and wherein the membrane is positioned intermediate between a distal end and a proximal end of the sequential stopper.

2. The sequential stopper of claim 1, wherein each of the proximal face and the distal face has a flat shape, a convex shape, a concave shape, or a multi-shape profile.

3. The sequential stopper of claim 1, wherein the plurality of circumferential ribs comprises:
a first rib extending radially outward from an outer surface of the lateral wall and around an outer circumference thereof, the first rib comprising a distal rib; and
a second rib extending radially outward from the outer surface of the lateral wall and around the outer circumference thereof, the second rib comprising a proximal rib;
wherein the first rib is spaced apart lengthwise from the second rib by an inter-rib region of the lateral wall.

4. The sequential stopper of claim 3, wherein the inter-rib region comprises a curved inter-rib region, where an outer surface of the lateral wall is curved radially inward.

5. The sequential stopper of claim 4, wherein the curved inter-rib region has a radius of curvature of 1 mm to 10 mm.

6. The sequential stopper of claim 5, wherein the radius of curvature is between 2 mm and 3 mm.

7. The sequential stopper of claim 3, wherein an inner surface of the lateral wall joins with the membrane via connecting portions therebetween, with the connecting portions providing a hinge about which the membrane deflects or pivots when pressure is exerted onto the proximal face of the membrane by the composition that causes the slit to open.

8. The sequential stopper of claim 7, wherein a lengthwise region between the proximal rib and the membrane and between the distal rib and the membrane comprises a force transfer area, where a radially inward-directed compressive force from the syringe barrel pressing against the first and second ribs is transferred to the membrane, to keep the slit closed under radial compression.

9. The sequential stopper of claim 8, wherein an angle of force transfer from the first and second ribs to the membrane, via the lateral wall and connecting portions, is between 20 degrees and 60 degrees, and preferably between 30 degrees and 45 degrees.

10. The sequential stopper of claim 7, wherein a thickness of the lateral wall and connecting portions in the force transfer area is between 0.5 mm and 2 mm.

11. The sequential stopper of claim 1, wherein the plurality of ribs comprises:
a first rib extending radially outward from an outer surface of the lateral wall and around an outer circumference thereof, the first rib comprising a distal rib;
a second rib extending radially outward from the outer surface of the lateral wall and around the outer circumference thereof, the second rib comprising a proximal rib; and
a third rib extending radially outward from the outer surface of the lateral wall and around the outer circumference thereof, the third rib comprising a center rib;
wherein the membrane is aligned with the third rib lengthwise along the sequential stopper.

12. The sequential stopper of claim 11, wherein the first rib and the second rib have a first outer diameter and the third rib has a second outer diameter that is smaller than the first outer diameter.

13. The sequential stopper of claim 1, wherein the membrane comprises one or more gutters formed therein, in the proximal face and/or the distal face of the membrane, with the one or more gutters reducing the thickness of the membrane at a location thereof, and wherein the membrane deflects or pivots about the one or more gutters when pressure is exerted onto the proximal face of the membrane by the composition that causes the slit to open.

14. The sequential stopper of claim 1, wherein the sequential stopper is a mono-material stopper, with the membrane and lateral wall formed as a single molded component.

15. The sequential stopper of claim 1, wherein the sequential stopper is a bi-material stopper, with the membrane formed of a first material and the lateral wall formed of a second material.

16. A medical injection device for injecting at least one fluid, comprising:
a barrel extending from a proximal end to a distal end, the barrel comprising a cylindrical wall, an end wall positioned at the distal end, and a tip extending distally from the end wall;
a plunger stopper adapted to be translationally movable inside the barrel; and
the sequential stopper of claim 1, arranged between the distal end of the barrel and the plunger stopper, and adapted to be translationally movable inside the barrel, wherein the lateral wall of the sequential stopper sealingly engages the inner surface of the barrel;
wherein a first chamber is defined within the barrel between the distal end of the barrel and the sequential stopper and a second chamber is defined within the barrel between the sequential stopper and the plunger stopper.

17. The medical injection device of claim 16, wherein the medical injection device is configured to sequentially inject two fluids, with a first fluid contained within the first chamber and a second fluid contained within the second chamber, and wherein the sequential stopper is spaced apart distally from the end wall to separate the first chamber from the second chamber.

18. The medical injection device of claim 16, wherein the sequential stopper comprises a first sequential stopper, and wherein the medical injection device further comprises a second sequential stopper, the second sequential stopper identical to the first sequential stopper, with the first sequential stopper and the second sequential stopper in part defining a first chamber, a second chamber, and a third chamber in the barrel.

19. The medical injection device of claim 18, wherein the medical injection device is configured to sequentially inject three fluids, with a first fluid contained within the first chamber, a second fluid contained within the second chamber, and a third fluid contained within the third chamber.

20. The medical injection device of claim 16, being one of a syringe or a cartridge.
